(19) United States
(12) Patent Application Publication
(10) Pub. No.: US 2016/0287777 A1
(43) Pub. Date: Oct. 6, 2016

(54) Method and Apparatus for Producing Platelet Rich Plasma

(71) Applicant: Alexander Nemirovsky, Santa Monica, CA (US)

(72) Inventor: Alexander Nemirovsky, Santa Monica, CA (US)

(21) Appl. No.: 15/182,506

(22) Filed: Jun. 14, 2016

Related U.S. Application Data
(62) Division of application No. 14/474,504, filed on Sep. 2, 2014, now abandoned.

(51) Int. Cl. A61M 1/34; A61M 1/36; B04B 5/04; B01L 3/00

(52) U.S. Cl. CPC A61M 1/3496; B01L 3/5021; A61M 1/3693; A61M 1/3673; B04B 5/0414

(57) ABSTRACT

A device and method for producing a portion of platelet rich plasma from a fluid sample obtained from a patient. The device comprises three intercommunicated portions, each portion having an internal chamber defined therein. The fluid sample is first brought into the base portion where it is separated into red blood cells and plasma. The plasma portion of the sample is then withdrawn into the second portion where it is further separated into platelet poor plasma disposed over platelet rich plasma. The portion of platelet poor plasma is then withdrawn from the second chamber, leaving only platelet rich plasma within the second chamber. The base portion also includes a membrane located on its distal end which helps maintain a vacuum within the device when the fluid sample is being initially inserted into base chamber.

### RELATED APPLICATIONS

The present application is related to U.S. Provisional Patent Application Ser. No. 61/873,207, filed on Sep. 3, 2013, and is a divisional of U.S. Nonprovisional patent application Ser. No. 14/474,504, filed on Sep. 2, 2014, both of which are incorporated herein by reference and to which priority is claimed pursuant to 35 USC 120.

### BACKGROUND

1. Field of the Technology

The disclosure relates to the field of producing therapeutic agent specifically a device and method for producing a platelet rich plasma.

2. Description of the Prior Art

Platelet rich plasma (PRP) is blood plasma that has been enriched with a concentrated source of platelets. PRP is commonly used as part of a therapeutic treatment regimen to rehabilitate joint and soft tissue (Brian J. Cole, MD, MBA, et al. Platelet-Rich Plasma: Where Are We Now and Where Are We Going? Sports Health 2010; vol. 2, 3: 203-100). In order to be the most effective, therapeutic PRP requires a platelet concentration of roughly 1.5 million platelets per micro liter (Giusti, I et al. Identification of an optimal concentration of platelet gel for promoting angiogenesis in human endothelial cells. Transfusion 2009; 49: 771-78).

Several methods for producing platelet rich plasma have been used previously by those skilled in the art to various levels of success. The basic principle underlying most of these previous techniques has been to draw a sample of blood from a patient into a syringe or other similar vessel. The vessel is then spun within a centrifuge, which separates the blood sample into red blood cells and PRP, or red blood cells, PRP, and platelet poor plasma (PPP) depending upon the specific configuration of the vessel and preparation technique. Most commonly, the vessel comprises multiple inner channels or compartments, which house each of the various components of the patient's blood sample. PPP and red blood cells are then removed from the device leaving the PRP behind and available for use as therapeutic treatment. Because the PRP was produced from the blood sample which originated with the patient who is now receiving therapeutic treatment, there is no risk of the patient's body rejecting the PRP and not accepting the treatment.

A problem exists however in producing PRP of high enough concentrations which make it appropriate for specific tissue treatment. Many previously known methods and devices produce PRP with platelet concentrations too low to provide an effective therapeutic agent. Additionally, many known methods and devices which produce PRP are difficult to use and require multiple clumsy or awkward steps in order to efficiently produce a viable therapeutic agent which increases the chances of the sterility of the product being compromised. Furthermore, many manufacturers of commercial kits do not disclose the concentration of platelets achievable with their kits, thus introducing some amount of uncertainty into the procedure. What is needed is a new method and device, which is capable of producing PRP at extremely high concentrations suitable for use as a therapeutic agent that is safe, efficient, and easy to use at the patient's point of care.

### BRIEF SUMMARY

The invention provides a device for producing platelet rich plasma (PRP). The device has base portion with a base chamber inside, and a second portion connected to the base portion, the second portion having a middle chamber inside of it. A third portion is also connected to the second portion, the third portion having a top chamber defined therein. The middle chamber of the device is fluidly communicated to the base chamber, and the top chamber is in turn fluidly communicated to the middle chamber.

In one embodiment, the second portion and the third portion are removably coupled from the base portion and the second portion, respectively.

In another embodiment, the device has a base plunger disposed within the base chamber and a nozzle disposed on the second portion which is capable of being removably coupled to the base plunger. A middle plunger is in turn disposed within the middle chamber and a secondary nozzle is located on the third portion which is configured to removably couple to the middle plunger.

In another embodiment, the base portion also includes an aperture disposed on its distal end which is configured to accommodate a needle or fluid line.

In a separate embodiment, a distal end of the base portion is made of a membrane.

In yet another embodiment, the base chamber is pre-filled with an anticoagulant agent.

Additionally, in a different embodiment the third portion of the device includes a plunger rod disposed within the top chamber.

The invention also includes a device for producing platelet rich plasma (PRP) which includes a base portion with a base chamber defined therein and a second portion that is connected to the base portion, the second portion having a middle chamber defined therein. The device further has a membrane on a distal end of the base portion and an anticoagulant disposed within the base chamber in a vacuum. The device is further characterized in that the middle chamber is fluidly communicated to the base chamber.

The invention also includes a method of producing a platelet rich plasma (PRP). The method includes obtaining a fluid sample from a patient and disposing the fluid sample into a first chamber. The fluid sample is then separated into a portion of red blood cells and a portion of plasma. The portion of plasma is then removed from the portion of red blood cells. The portion of plasma is itself then separated into a portion of platelet poor plasma and a portion of platelet rich plasma. Finally, the portion of platelet poor plasma is isolated from the portion of platelet rich plasma.

In one embodiment, the method step of removing the portion of plasma from the portion of red blood cells includes withdrawing the portion of plasma from the first chamber containing the fluid sample and disposing it into a second chamber which is fluidly communicated to the first chamber, leaving the portion of red blood cells in the first chamber, and then decoupling the second chamber from the first chamber. In this embodiment, withdrawing the portion of plasma from the first chamber and disposing it in the second chamber may be accomplished by increasing an internal volume of the second chamber.

In a related embodiment, the method step of isolating the platelet poor plasma from the platelet rich plasma includes withdrawing the portion of platelet poor plasma from the second chamber and disposing it into a third chamber which is fluidly communicated to the second chamber, leaving the portion of platelet rich plasma in the second chamber, and then decoupling the third chamber from the second chamber. In this embodiment, withdrawing the portion of platelet poor plasma from the second chamber and disposing it into the third chamber may be accomplished by increasing an internal volume of the third chamber.

In another embodiment, the method step of separating the fluid sample into a portion of red blood cells and a portion of plasma includes disposing the fluid sample in a centrifuge and then spinning the fluid sample in the centrifuge for a predetermined amount of time at a predetermined speed. A portion of plasma and a portion of red blood cells is then formed within a first chamber, the portion of plasma having moved to a position above the portion of red blood cells.

In a related embodiment, the method step of separating the portion of plasma into a portion of platelet poor plasma and a portion of platelet rich plasma includes disposing the portion of plasma in a centrifuge and then spinning the portion of plasma in the centrifuge for a predetermined amount of time at a predetermined speed. A portion of platelet poor plasma and a portion of platelet rich plasma is then formed within a second chamber, the portion of platelet poor plasma having moved to a position above the portion of platelet rich plasma.

In another embodiment, the method step of obtaining a fluid sample from a patient and disposing the fluid sample into a first chamber includes connecting a needle or a fluid line to an aperture connected to the first chamber.

In yet another embodiment, the method step of obtaining a fluid sample from a patient and disposing the fluid sample into a first chamber includes inserting a needle through a membrane located on a distal end of the first chamber.

The invention also includes a method of producing a platelet rich plasma in a device. First, a fluid sample is obtained from a patient and is then disposed into a first chamber of the device which is vacuum sealed. The fluid sample is then separated into a portion of red blood cells and a portion of plasma. The portion of plasma is then removed from the portion of red blood cells and is disposed into a second chamber. Next, the portion of plasma is separated into a portion of platelet poor plasma and a portion of platelet rich plasma. Finally, the portion of platelet poor plasma is isolated from the portion of platelet rich plasma by disposing the portion of platelet poor plasma in a third chamber.

In one embodiment, the method step of obtaining a fluid sample from a patient and disposing the fluid sample into a first chamber of the device includes inserting a needle containing the fluid sample through a membrane located on a distal end of the first chamber.

In another embodiment, the method step of obtaining a fluid sample from a patient and disposing the fluid sample into a first chamber of the device further includes mixing the fluid sample with an anticoagulant disposed within the first chamber.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

FIGS. 1A and 1Ai are frontal views of two embodiments of the device with a needle aperture and a membrane disposed on the distal end of the device, respectively.

FIGS. 1B and 1Bi are cross sectional views of the two embodiments of the device seen in FIGS. 1A and 1Ai, respectively.

FIG. 2A is a schematic cross sectional diagram of the device showing the base, middle, and top portions of the device being coupled together.

FIG. 2B is a schematic cross sectional diagram of the device with the fluid sample obtained from the patient disposed in the base portion.

FIG. 2C is a schematic cross sectional diagram of the device after the fluid sample has been separated into red blood cells and plasma within the base portion.

FIG. 2D is a schematic cross sectional diagram of the device as the plasma portion of the fluid sample is being withdrawn into the middle portion of the device.

FIG. 2E is a schematic cross sectional diagram of the device after the base portion has been decoupled from the middle portion, leaving the plasma disposed within the middle portion.

FIG. 2F is a schematic cross sectional diagram of the device after the plasma has been separated into platelet poor plasma and platelet rich plasma within the middle portion.

FIG. 2G is a schematic cross sectional diagram of the device as the portion of platelet poor plasma is being withdrawn into the top portion of the device.

FIG. 2H is a schematic cross sectional diagram of the device after the top portion has been decoupled from the middle portion, leaving the platelet rich plasma disposed within the middle portion.

FIG. 3 is a schematic cross sectional diagram of an alternative embodiment of the device seen in FIG. 1Ai where the base portion comprises a membrane disposed on its distal end and an anticoagulant disposed therein.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

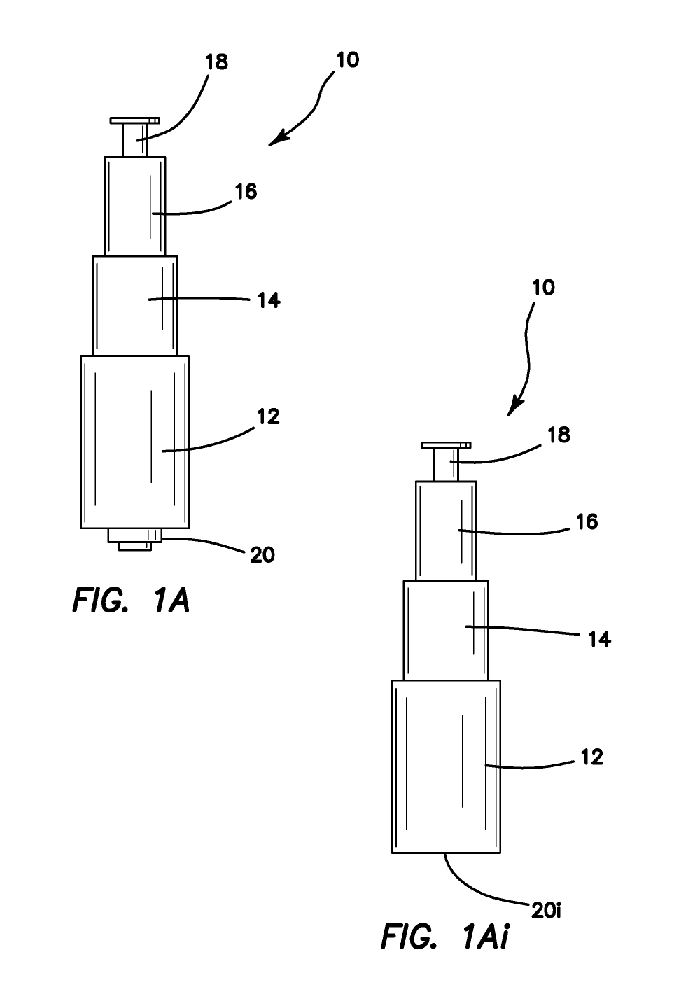

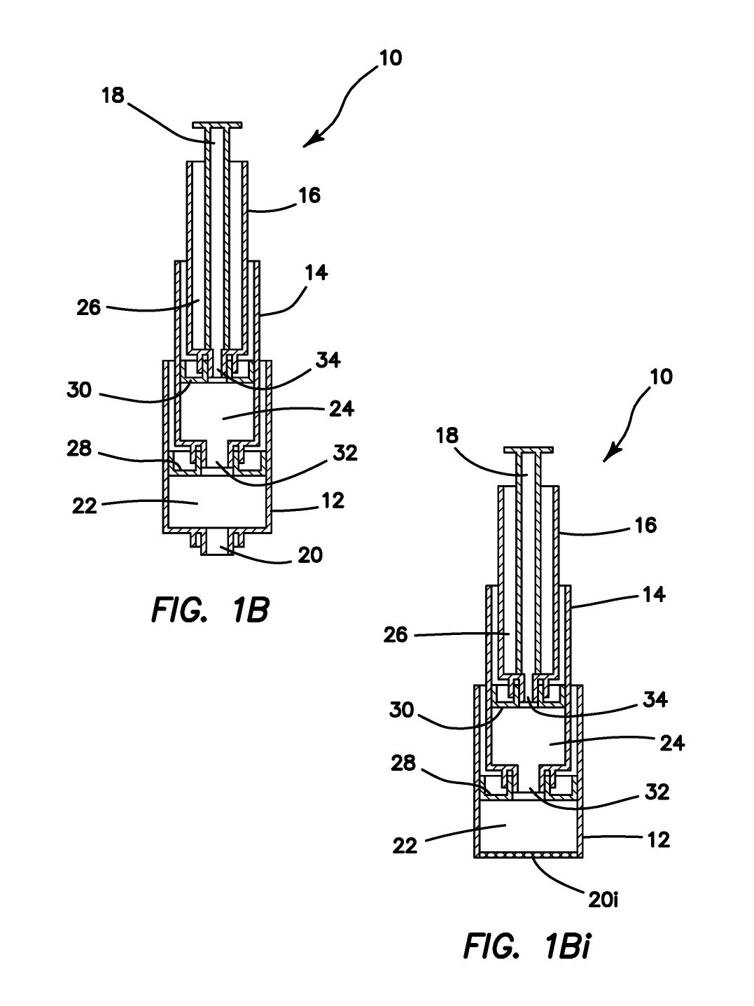

A greater understanding of the current invention may be had by turning to FIGS. 1A, 1Ai, 1B and 1Bi where the device is generally denoted with reference numeral 10. The device 10 comprises a substantially tubular-like-compartment configuration with a tiered or staged design. The devise exists in two versions—first is for the user who is planning to prepare PRP according to his/her own protocol as seen in FIG. 1A (various amount of blood drawn from the patient, different or no anticoagulant, etc.); the second is for the user who is planning to adhere to the protocol suggested in this invention (FIG. 1Ai). These two versions are very similar except for the bottom surface of the outer tube 12. The embodiment seen in FIG. 1Ai has an elastic membrane 20i which is rigid enough to maintain a vacuum inside the tube 12 and at the same time elastic enough to allow a needle of a blood drawing line to penetrate it. In this embodiment, a predetermined amount of blood will enter the tube 12 during blood draw which contains a prefilled amount of anticoagulant. The user is discouraged from opening the end of a tube 12 prior to the completion of the first step of the separation process as is detailed further below. In addition to offering simplicity, the embodiment seen in FIG. 1Ai helps the operator to maintain complete sterility without significant effort on the part of the operator.

In contrast, the embodiment seen in FIG. 1A has an aperture with a one-way valve or Luer lock 20, which may accommodate or couple to a needle or fluid line. With this embodiment, a user can draw any amount of blood (not exceeding the size of the container) and use any type of anticoagulant or no anticoagulation at all. The user is encouraged to exercise extreme caution when using this version of the device in order to maintain its sterility.

The device 10 comprises a base portion 12, a middle portion 14, and a top portion 16. The three portions 12, 14, 16 are nested within one another as best seen in the cross sectional view of FIGS. 1B and 1Bi, with each portion or stage inserted into the center of the portion disposed directly beneath it. The three portions 12, 14, 16 also share a common central longitudinal axis. The top portion 16 comprises a plunger rod 18 disposed through its center. The base portion 12 as mentioned above comprises either an elastic (rubber type) membrane 20i or a needle aperture 20 with a one-way valve or Luer lock. The former will allow a needle to penetrate it during blood draw, the latter may accommodate or couple to a needle or fluid line as is known in the art.

The base portion 12 of the device 10 comprises an internal base chamber 22 and an internal base plunger 28 as seen in FIGS. 1B and 1Bi. The base plunger 28 is slidable within the base chamber 22 so that it may traverse the internal longitudinal surface of the base chamber 22. The distal end of the middle portion 14 comprises a nozzle 32 with means for fitting into the base plunger 28 by compression fit, friction fit, or other well-known means in the art. The nozzle 32 allows fluidic communication between the base chamber 22 and the corresponding middle chamber 24 defined within the middle portion 14.

The middle portion 14 further comprises a corresponding internal middle plunger 30 which is slidably disposed within the middle chamber 24. A secondary nozzle 34 disposed on the distal end of the top portion 16 comprises means for being temporarily coupled to the middle plunger 30 via a compression fit, friction fit, or other equivalent means known in the art. The secondary nozzle 34 allows fluidic communication between the middle chamber 24 and the top chamber 26 defined within the top portion 16. The top portion 16 further comprises the plunger rod 18 which is disposed through the longitudinal length of the top portion 16 as seen in FIGS. 1B and 1Bi.

When the top portion 16 is coupled to the middle plunger 30 of the middle portion 14, and when the middle portion 14 itself is coupled to the base plunger 28 of the base portion 12, the device 10 forms a substantially stacked or tiered configuration as seen in FIGS. 1A and 1B. The device 10 is ready for use when in the stacked configuration and may be assembled by the user or alternatively may reach the user already assembled within sterile packaging or a sterilized kit.

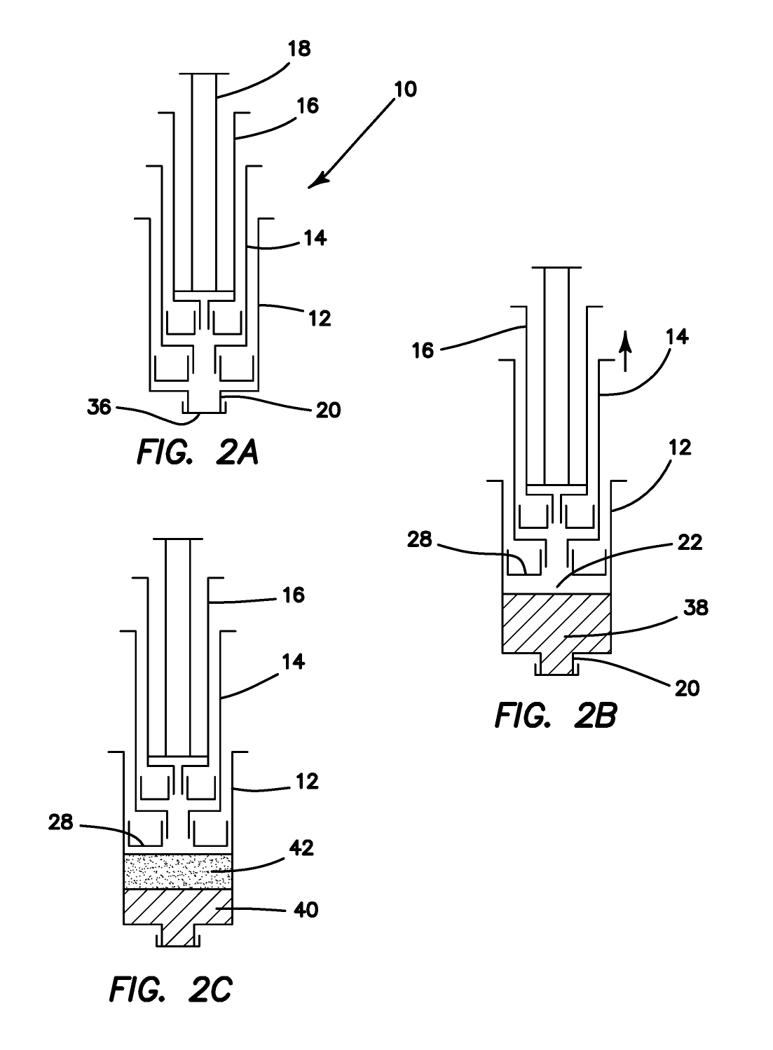

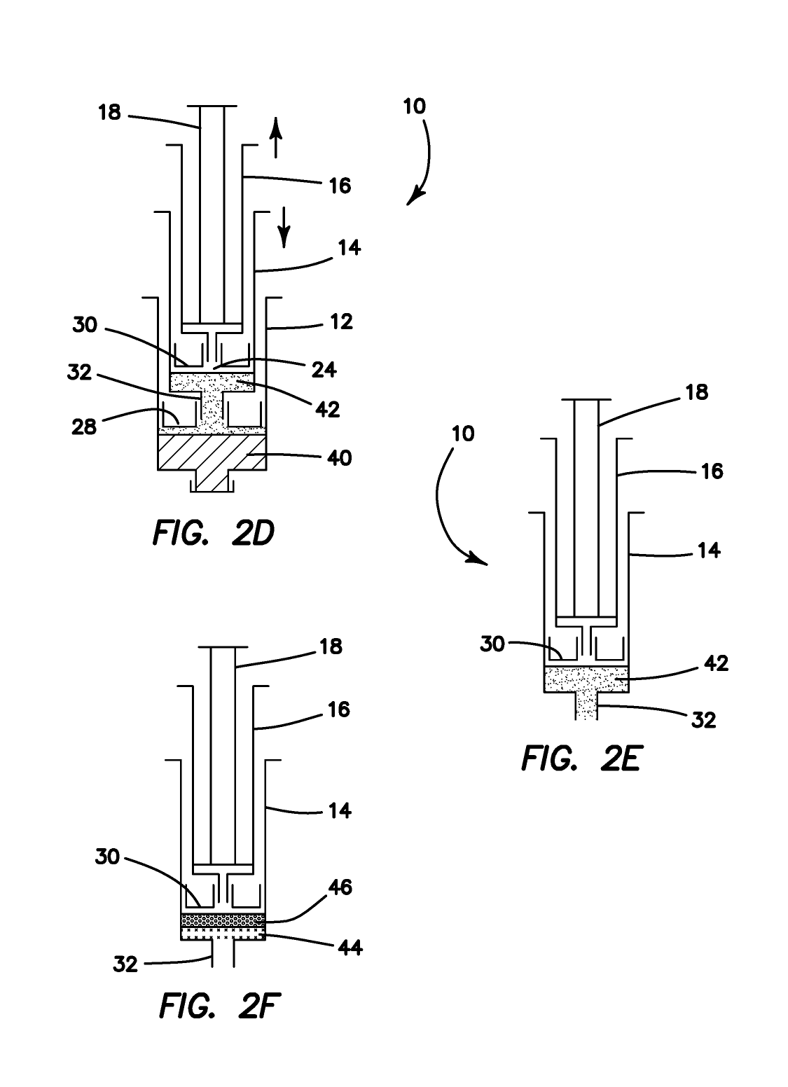

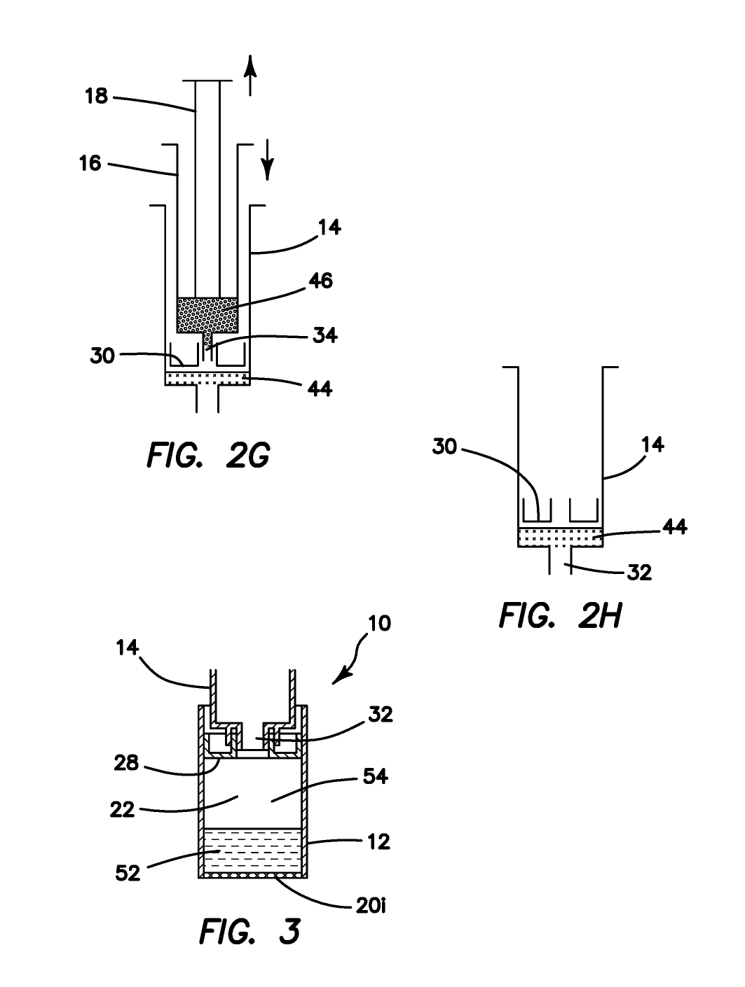

The operation of the two embodiments of the device is described separately. The first embodiment of the device 10 comprising a needle aperture shown on FIGS. 1A and 1B may be used as shown in FIGS. 2A-2H. In FIG. 2A, the device 10 is seen with all three portions 12, 14, 16 in the stacked configuration. A needle or an incoming fluid line 36 is coupled to the aperture 20. Prior to blood draw the container can be prefilled with an anticoagulant of the operators choice, or alternatively, no anticoagulant may present depending upon the operator's specific needs. The middle portion 14 is then pulled upward in the direction of the arrow shown in FIG. 2B, drawing a fluid sample 38 from a patient through the fluid line 36 and into the internal base chamber 22 of the base portion 12. The fluid sample 38 fills a substantial volume of base chamber 22 from the aperture 20 to the bottom surface of the base plunger 28.

After obtaining the entirety of the fluid sample 38 from the patient, the device 10 is spun in a centrifuge for approximately ten minutes at 112 g. Alternatively, the device 10 may also be spun for four minutes at 252 g, or for two minutes at 447 g according to need and user preference. It should also be noted that other combinations or products of time and relative centrifugal force may be used without departing significantly spirit and scope of the original invention. After being spun, the fluid sample 38 separates into a portion of red blood cells (RBC), and a portion of plasma. Because of centrifugal forces, the heavier red blood cells 40 travel to and accumulate at the bottom of the base chamber 22 as seen in FIG. 2C, while the lighter plasma portion 42 collects at the top of the base chamber 22.

The device 10 is then removed from the centrifuge and then, as seen in FIG. 2D, the middle portion 14 is pushed downward in the direction shown by the arrow next to the middle portion 14 in the figure. At the same time, the top portion 16 is pulled upwards in the direction shown by the arrow next to the top portion 16 in the figure. Pushing the middle portion 14 downwards into the bottom portion 12 decreases the volume of the bottom chamber 22 and squeezes the plasma 42 through the nozzle 32 and into the middle chamber 24. At the same time, pulling the top portion 16 upwards simultaneously increases the volume of the middle chamber 24, further drawing the plasma 42 into the middle portion 14. As the plasma 42 is being drawn into the middle chamber 24, the bottom chamber 22 continually contracts leaving only the RBCs 40 in the bottom portion 12. The middle portion 14 is then decoupled from the bottom portion 12, with the nozzle 32 disengaging from the bottom plunger 28 by means known in the art, leaving the middle portion 14 and top portion 16 intact as seen in FIG. 2E. The nozzle 32 comprises a one-way valve or Luer lock which keeps the plasma 42 within the middle chamber 24. The bottom portion 12 containing the RBCs 40 is then either discarded or properly stored for another purpose.

At this point, the operator or user may stop the process if he or she is satisfied with the platelet concentration of the plasma 42. If so, the plasma 42 may then be ejected from the middle portion 14 straight into the treatment site of the patient. If the user desires a higher platelet concentration, the user continues further as outlined below.

After the bottom portion 12 has been removed as seen in FIG. 2E, the device 10 is placed back into the centrifuge and is then spun at 1000 g for an additional 10 minutes. After spinning, the device 10 is removed from the centrifuge and, as is seen in FIG. 2F, the plasma 42 has been further divided into two component fluids, specifically—a platelet rich plasma (PRP) 44 and a platelet poor plasma (PPP) 46. Because of gravity and centrifugal forces applied by the centrifuge, the PRP 44 travels down to the bottom of the middle chamber 24 while the lighter, less dense PPP 46 remains near the top of the middle chamber 24 above the PRP 44.

In order to isolate the PRP 44, the plunger rod 18 of the top portion 16 is pulled upward in the direction seen by the arrow in FIG. 2G. Pulling upward on the plunger rod 18 increases the internal volume of the top portion 16 which in turn draws the PPP 46 through the secondary nozzle 34 and up into the top portion 16. The secondary nozzle 34 is fluidly communicated to the PPP 46 and the user continues to pull the plunger rod 18 upward or alternatively push the top portion 16 downward until all the PPP 46 is removed from the middle portion 14. The top portion 16 is then decoupled from the middle portion 14 with the secondary nozzle 34 disengaging from the middle plunger 30 by means known in the art, leaving the middle portion 14 intact and containing only PRP 44 as seen in FIG. 2H. The top portion 16 containing the patient's PPP 46 is either then discarded or properly stored for another use or application.

With the PRP 44 isolated in the middle portion 14, the user is then free to store as needed or immediately begin treatment of the patient by re-injecting the PRP 44 into the patient according to their prescribed treatment regimen. Because the PRP 44 is produced by the device 10 in the manner described, the PRP 44 may comprise platelet concentrations of at least one million platelets per micro liter. It should be noted that the above specified platelet concentration is for illustrative purposes only and that additional or different specific concentrations may be possible using the current device 10 without departing from the original spirit and scope of the invention.

In another embodiment, the middle portion 14 and nested top portion 16 are placed in the centrifuge upside down, with the nozzle 32 facing upwards and the middle plunger 30 facing downwards. In this configuration, after spinning the heavier PRP 44 will travel and settle next to the middle plunger 30 while the PPP 46 will settle next to the nozzle 32. In this embodiment, the user simply separates the PPP 46 from the PRP 44 by pushing the plunger 30 and expelling the PPP 46 from the nozzle 32 and leaving the PRP 44 within the middle chamber 24 of the middle portion 14. In this embodiment, the top portion 16 is not needed and the user may manipulate the bottom and middle portions 12, 14 as needed, leaving the top portion 16 as an optional structural component.

The second version of apparatus 10, which reduces possible contamination of the fluid sample 38 is seen in FIG. 3. On the distal end of the base portion 12, there is an elastic cap 20i disposed which is comprised of rubber, plastic, or other known materials, which are known to be rigid enough to maintain a vacuum and yet soft enough to be penetrated by a needle during the blood draw. On the proximal end of the base portion 12 there is an attachment (middle portion) 14. The distal end of the middle portion 14 comprises a nozzle 32 with means for fitting into the base plunger 28 by compression fit, friction fit, or other well-known means in the art. The attachment 14 in this embodiment is locked into place to maintain a vacuum 54 within the base portion 12. The base portion 12 is prefilled with an anticoagulant 52 within the vacuum 54. The amount of fluid sample 38 drawn into the chamber 22 is predetermined by the size of the chamber.

Up to this point, sterility of the container 12 is maintained and contamination is almost impossible. If the blood is collected into a standard syringe, then it becomes necessary to open a syringe, draw anticoagulant 52 from a separate container, and then draw blood into the syringe. During this procedure the sterility of the syringe could be compromised and contamination could occur.

After obtaining the fluid sample, the device 10 is placed into a centrifuge for the separation step as discussed above. After the separation, the attachment 14 is unlocked and the plasma is drawn into the attachment 14, which is later disconnected from the base portion 12. The plasma can then be used either for injection or placed back into the centrifuge for a second spin. During this procedure, the whole system is closed at all times thus keeping the risk of contamination to a minimum.

It should be apparent that in either embodiment discussed above, the user may alter the device 10 to suit a specific purpose. For example, the device 10 may comprise two or three stages depending upon the technique the operator would like to use without departing from the original spirit and scope of the invention. For example, the device 10 may selectively be spun either once or twice depending on what is required.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what s specifically illustrated and described above, what is conceptionally equivalent, what can be obviously substituted and also what essentially incorporates the essential idea of the embodiments.

I claim:

1. A method of producing a platelet rich plasma (PRP) comprising:
obtaining a fluid sample from a patient and disposing the fluid sample into a first chamber within a bottom portion of a stacked configuration device;
separating the fluid sample into a lower portion of red blood cells and an upper portion of plasma in the first chamber by centrifuging the stacked configuration device for a first predetermined time at a speed corresponding to a first predetermined g-force;
removing the upper portion of plasma from the lower portion of red blood cells by disposing a middle portion of the stacked configuration device into the upper portion of plasma in the first chamber and withdrawing the plasma into a middle chamber of the stacked configuration device leaving only red blood cells in the first chamber;
separating the portion of plasma into an upper portion of platelet poor plasma and a lower portion of platelet rich plasma in a second chamber of the stacked configuration device by centrifuging the stacked configuration device for a second predetermined time at a speed corresponding to a second predetermined g-force; and
isolating the upper portion of platelet poor plasma from the lower portion of platelet rich plasma by disposing an upper portion of the stacked configuration device into the upper portion of platelet poor plasma in the second chamber of the stacked configuration device and withdrawing the upper portion of platelet poor plasma into an upper chamber of the stacked configuration device leaving only the lower portion of platelet rich plasma in the second chamber.

2. The method of claim 1 where obtaining a fluid sample from a patient and disposing the fluid sample into a first chamber comprises coupling a needle or a fluid line to an aperture communicating with the first chamber.

3. The method of claim 1 where obtaining a fluid sample from a patient and disposing the fluid sample into a first chamber comprises:
creating a partial vacuum within the first chamber by withdrawing a plunger in the stacked configuration device and temporarily locking the plunging into position to maintain the partial vacuum; and
inserting a needle through a fluid-tight membrane disposed on a distal end of the first chamber and disposing a predetermined amount of the fluid sample into the first chamber through the needle.

4. The method of claim 1 where separating the fluid sample into a lower portion of red blood cells and an upper portion of plasma in the first chamber by centrifuging the stacked configuration device for a first predetermined time at a speed corresponding to a first predetermined g-force comprises centrifuging the stacked configuration device for at least 10 minutes at a speed corresponding to a first predetermined g-force of at least 112 g.

5. The method of claim 1 where separating the fluid sample into a lower portion of red blood cells and an upper portion of plasma in the first chamber by centrifuging the stacked configuration device for a first predetermined time at a speed corresponding to a first predetermined g-force comprises centrifuging the stacked configuration device for at least 4 minutes at a speed corresponding to a first predetermined g-force of at least 252 g.

6. The method of claim 1 where separating the fluid sample into a lower portion of red blood cells and an upper portion of plasma in the first chamber by centrifuging the stacked configuration device for a first predetermined time at a speed corresponding to a first predetermined g-force comprises centrifuging the stacked configuration device for at least 2 minutes at a speed corresponding to a first predetermined g-force of at least 447 g.

7. The method of claim 1 where separating the portion of plasma into an upper portion of platelet poor plasma and a lower portion of platelet rich plasma in a second chamber of the stacked configuration device by centrifuging the stacked configuration device for a second predetermined time at a speed corresponding to a second predetermined g-force comprises centrifuging the stacked configuration device for at least 10 minutes at a speed corresponding to a second predetermined g-force of at least 1000 g.

8. The method of claim 1 where obtaining a fluid sample from a patient and disposing the fluid sample into a first chamber of the device further comprises mixing the fluid sample with an anticoagulant predisposed within the first chamber.

9. A method of producing a platelet rich plasma (PRP) comprising:
obtaining a fluid sample from a patient and disposing the fluid sample into a first chamber within a bottom portion of a stacked configuration device;
separating the fluid sample into a lower portion of red blood cells and an upper portion of plasma in the first chamber by centrifuging the stacked configuration device for a first predetermined time at a speed corresponding to a first predetermined g-force;
removing the upper portion of plasma from the lower portion of red blood cells by disposing a middle portion of the stacked configuration device into the upper portion of plasma in the first chamber and withdrawing the plasma into a middle chamber of the stacked configuration device leaving only red blood cells in the first chamber;
separating the portion of plasma into an upper portion of platelet poor plasma and a lower portion of platelet rich plasma in a second chamber of the stacked configuration device by inverting the stacked configuration device and by centrifuging the inverted stacked configuration device for a second predetermined time at a speed corresponding to a second predetermined g-force; and
isolating the upper portion of platelet poor plasma from the lower portion of platelet rich plasma in the second chamber by expelling the portion of platelet poor plasma from the second chamber of the stacked configuration device leaving only the portion of platelet rich plasma the second chamber of the stacked configuration device.

10. The method of claim 9 where obtaining a fluid sample from a patient and disposing the fluid sample into a first chamber comprises coupling a needle or a fluid line to an aperture communicating with the first chamber.

11. The method of claim 9 where obtaining a fluid sample from a patient and disposing the fluid sample into a first chamber comprises:
creating a partial vacuum within the first chamber by withdrawing a plunger in the stacked configuration device and temporarily locking the plunger into position to maintain the partial vacuum; and
inserting a needle through a fluid-tight membrane disposed on a distal end of the first chamber and disposing a predetermined amount of the fluid sample into the first chamber through the needle.

12. The method of claim 9 where separating the fluid sample into a lower portion of red blood cells and an upper portion of plasma in the first chamber by centrifuging the stacked configuration device for a first predetermined time at a speed corresponding to a first predetermined g-force comprises centrifuging the stacked configuration device for at least 10 minutes at a speed corresponding to a first predetermined g-force of at least 112 g.

13. The method of claim 9 where separating the fluid sample into a lower portion of red blood cells and an upper portion of plasma in the first chamber by centrifuging the stacked configuration device for a first predetermined time at a speed corresponding to a first predetermined g-force comprises centrifuging the stacked configuration device for at least 4 minutes at a speed corresponding to a first predetermined g-force of at least 252 g.

14. The method of claim 9 where separating the fluid sample into a lower portion of red blood cells and an upper portion of plasma in the first chamber by centrifuging the stacked configuration device for a first predetermined time at a speed corresponding to a first predetermined g-force comprises centrifuging the stacked configuration device for at least 2 minutes at a speed corresponding to a first predetermined g-force of at least 447 g.

15. The method of claim 9 where separating the portion of plasma into an upper portion of platelet poor plasma and a lower portion of platelet rich plasma in a second chamber of the stacked configuration device by centrifuging the stacked configuration device for a second predetermined time at a speed corresponding to a second predetermined g-force comprises centrifuging the stacked configuration device for at least 10 minutes at a speed corresponding to a second predetermined g-force of at least 1000 g.

16. The method of claim 9 where obtaining a fluid sample from a patient and disposing the fluid sample into a first chamber of the device further comprises mixing the fluid sample with an anticoagulant predisposed within the first chamber.

17. A method of producing a platelet rich plasma (PRP) comprising:
creating a partial vacuum within a first chamber sealed with a self-sealing membrane in a stacked configuration device and temporarily maintaining the partial vacuum in the first chamber; and
disposing a predetermined amount of a fluid sample of blood obtained from a patient into the first chamber using a needle inserted through the self-sealing membrane thereby insuring the maintenance of sterility;
separating the fluid sample of blood into a portion of red blood cells and a portion of plasma by centrifuging the stacked configuration device;
removing the portion of plasma from the portion of red blood cells within the stacked configuration device while maintaining sterility;
separating the portion of plasma into a portion of platelet poor plasma and a portion of platelet rich plasma by centrifuging the stacked configuration device; and
isolating the portion of platelet poor plasma from the portion of platelet rich plasma within the stacked configuration device while maintaining sterility.

18. The method of claim 17 where separating the fluid sample of blood into a portion of red blood cells and a portion of plasma by centrifuging the stacked configuration device comprises centrifuging the stacked configuration device for at least 10 minutes at a speed corresponding to a first predetermined g-force of at least 112 g.

19. The method of claim 17 where separating the fluid sample of blood into a portion of red blood cells and a portion of plasma by centrifuging the stacked configuration device comprises centrifuging the stacked configuration device for at least 4 minutes at a speed corresponding to a first predetermined g-force of at least 252 g.

20. The method of claim 17 where separating the fluid sample of blood into a portion of red blood cells and a portion of plasma by centrifuging the stacked configuration device comprises centrifuging the stacked configuration device for at least 2 minutes at a speed corresponding to a first predetermined g-force of at least 447 g.

21. The method of claim 17 separating the portion of plasma into a portion of platelet poor plasma and a portion of platelet rich plasma by centrifuging the stacked configuration device comprises centrifuging the stacked configuration device for at least 10 minutes at a speed corresponding to a second predetermined g-force of at least 1000 g.
